(19) United States
(12) Patent Application Publication
(10) Pub. No.: US 2007/0197876 A1
(43) Pub. Date: Aug. 23, 2007

(54) DENTAL GUARD FOR AIRWAY INTUBATION

(76) Inventor: Edward D. Lane, Cal-Nev-Ari, NV (US)

Correspondence Address: TRAVIS CHANDLER, PHD, LLC, 1489 WEST WARM SPRINGS ROAD, SUITE 110, HENDERSON, NV, 89014, US

(21) Appl. No.: 11/307,697

(22) Filed: Feb. 17, 2006

(51) Int. Cl. A61B 1/267

(52) U.S. Cl. 600/195

(57) ABSTRACT

The present invention relates to the field of medical devices used in orotracheal intubation. The insertion and manipulation of laryngoscopes used by medical personnel may occasionally cause damage or breakage of incisors or other maxillary teeth. The present invention provides a device useful for the prevention of tooth damage occurring with common laryngoscopes and other airway intubation systems. The device comprises an upper mouthguard for the maxillary teeth attached to blocks or wedges for keeping the jaw open. Optionally, a light such as an LED may be incorporated in one of the blocks to facilitate the intubation procedure. The invention is placed into the subject's mouth, and then intubation using a laryngoscope may be safely performed. Once intubation has been accomplished, and the laryngoscope has been removed, the dental guard of this invention may be retained in the patient's jaw as a bite block, or removed, or replaced with another type of bite block.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### FIELD OF THE INVENTION

The present invention relates to the field of medical devices used in the procedures of orotracheal intubation. More particularly, the present invention relates to a dental guard which is designed to distribute and dissipate both direct pressure and shear forces on the maxillary incisor teeth when the laryngoscope is placed in a patient's mouth and manipulated during intubation.

### BACKGROUND OF THE INVENTION

Endotracheal intubation procedures are commonly employed to secure a controlled airway and to deliver oxygen or anesthetic gases into the lungs of patients. These procedures are often performed by emergency response technicians rendering aid to victims in the field, or sometimes while in transit to hospital emergency rooms. They may also be performed as a preliminary measure before a surgery, thus enabling the anesthesiologist to administer anesthetic gases and other drugs while maintaining a stable delivery of oxygen to the patient's lungs throughout the surgical procedure. In other situations, intubation is used to provide a patient with a secure, direct airway when mechanical ventilation of the patient is indicated.

The conventional procedure for such intubation procedures is well-described elsewhere, such as by Cartledge in U.S. Patent Application 20040034281, and involves the visualization of the glottis and adjacent anatomic areas with a laryngoscope. A laryngoscope typically includes a handle containing a power supply and an attached blade section that serves to displace the tongue and allow direct visualization of the patient's larynx through the mouth. The technique of orotracheal intubation involves sighting the vocal cords and the insertion of an endotracheal tube in a patient who is usually anesthetized or unconscious. The laryngoscope blade is usually introduced laterally through the right side of the mouth, and then directed medially to exclude and retract the tongue for adequate visualization of the hypopharynx.

In an intubation, the head, neck, and shoulders of the patient are positioned so that the oral, pharyngeal, and laryngeal axes are optimally aligned. The laryngoscope is then inserted, preferably using the left hand, laterally from the right side of the patient's mouth in such a way as to avoid the incisor teeth and the tongue is deflected away from the lumen of the pharyngeal outlet by the tip of the laryngoscope blade. The epiglottis is sighted above the base of the tongue and, according to the type of laryngoscope blade used, the instrument is manipulated to expose the glottic opening. The endotracheal tube is then introduced through either the mouth or nose, and visually advanced between the vocal cords into the subglottic space. Once placement has been achieved, the laryngoscope blade is carefully removed to avoid either injury to oropharyngeal structures or unintentional displacement of the endotracheal tube.

Many types of laryngoscope blades have been developed, where each type is characterized by blade curvature or lack thereof, the point of such curvature if the blade is curved, and the flange structure of the blade. Three types of blades are most commonly used. An example of the first type of blade, characterized as a curved blade, is known in the art as the Macintosh blade. The Macintosh blade is advanced into the space between the base of the tongue and the pharyngeal surface of the epiglottis. Forward and upward movement of this blade stretches the hypoepiglottic ligament to cause the epiglottis to move upward to expose the glottic opening. Two other types of prominently used blades are the straight blade, known as the Jackson or Wisconsin blade, and the straight blade with a curved tip, known in the art as the Miller blade. The tip of these blades are passed beneath the laryngeal surface of the epiglottis and moved upwardly to elevate the epiglottis, thereby exposing the glottic opening.

During insertion of the laryngoscope, care must be taken to avoid pressure on the teeth and gums of the patient. Use of a gentle technique for inserting the laryngoscope, may lessen, but cannot totally remove the possibility of intubation trauma. However, poor dentition or suboptimal anatomy of the teeth, jaw, neck, or throat may leave the maxillary incisors at particularly significant risk during intubation by even the most experienced of technicians. During emergency response situations, either in the field by paramedics or in the hospital emergency rooms for example, such gentle techniques are often not realized.

When in use, a laryngoscope blade or more often its handle may rest against the incisor teeth of the maxilla, depending upon the individual patient's anatomy. Because the laryngoscope is necessarily formed of a hard, inflexible material, dental damage is a potential result of such intubation procedures. The risk of such dental injury is typically aggravated when the upper teeth are used as a fulcrum during insertion procedures, as they commonly are. In most cases it is the handle of the laryngoscope that contacts and damages the teeth.

Various methods have been proposed to minimize such dental injury. The prior art teaches that other protective shields have limited advantage in preventing dental injuries during intubation. The shields cover the maxillary incisors, yet provide only limited protection for the teeth. Such shields are prone to displacement by either the laryngoscope blade or other instrumentation. Furthermore, existing shields are relatively difficult to use, requiring multiple operator steps to secure the shields for use. The multiple steps required to secure existing shields may discourage their use by significantly increasing the time and effort required to achieve the desired intubation. Moreover, the bulk of the existing protective shields may obstruct the user's view into the hypopharynx.

U.S. Pat. No. 3,826,248 to Gobels presents a laryngoscope blade in which an elastic insert which is anchored within a mating opening in the tongue deflector by means of undercut grooves extending in cross and longitudinal directions relative to each other.

U.S. Pat. No. 4,583,527 to Musicant et al. teaches the placement of an elongated layer of soft, pliable plastic material which adheres to the upper surface of a sheath which is slidably and removably coupled to the flange or curved edge of the tongue deflector of a laryngoscope blade.

U.S. Pat. No. 5,065,738 to Van Dam provides for a flexible padded sheath, which is adhered along the length of a laryngoscope blade, covering substantially its entire outer surface.

U.S. Pat. No. 5,438,976 to Nash provides a self-adherent, deformable cushioning device wrapped around a laryngoscope blade along at least a portion of its length.

U.S. Pat. No. 5,776,053 to Dragisic et al. provides a laryngoscope blade with a cutout or recess in the vertical portion of the tongue deflector where a resilient insert is removably coupled at its proximal and distal ends.

U.S. Patent Application 20040034281 by Cartledge provides a laryngoscope blade with a removable disposable partially resilient insert on the upper blade surface.

All of these devices represent additions or modifications to the basic laryngoscope design. But the devices currently available do not satisfactorily address the need to protect a patient's teeth, as well as the needs of convenience and feasibility of use. Blade covers are cumbersome and slow to apply to the blade, and therefore are often impractical in emergency situations.

Moreover, a cushioning device should be easy to remove after use and should not leave a permanent residue on the blade or the patient's teeth or mouth. While blade covers and inserts currently available are generally used only once, a typical laryngoscope blade is reusable and is commonly sterilized in steam autoclaves between uses. Adhesives used in applying a blade cover to a blade often make it difficult to remove the blade cover and may leave a residue that is difficult to remove. Adhesive residues may become even more difficult to remove after the residue has been subjected to the heat and pressure of the sterilization process. In addition, any such residue on a reusable instrument may present a potential focus for infectious bacteria or other pathogenic organisms. Further, blade covers that surround the entire blade or entire flange occupy too much space in a patient's mouth, making manipulation of the laryngoscope blade and introduction of the endotracheal tube difficult.

Finally, the prior art that is directed to protecting teeth from damage by laryngoscopes involve significant complications to the manufacture and use of the said laryngoscopes, involving multiple parts, and in some cases spring loaded components or other complications that make manufacture more costly and technician use more prone to errors.

### BRIEF SUMMARY OF THE INVENTION

The present invention is directed to a dental guard that is designed to reduce the risk of dental injuries in the course of endotracheal intubation in patients. The inventive guard is compatible for use with any of the conventional laryngoscopes, in both their straight and curved-blade embodiments.

The dental guard of the present invention is comprised of a mouthguard for the maxillary (upper) teeth only, such as are commonly used in sports applications such as football or boxing, with the addition of trapezoidal blocks attached at the posterior end of the guard, that is, the end that covers and protects the maxillary premolar and molar teeth. The mouthguard element is in the form of a U-shaped trough that embraces both the inside and outside of the teeth. The trapezoidal block elements extend downward to the mandibular teeth and are broad enough so that they rest securely on the mandibular molar teeth. They thereby assist the medical personnel in keeping the jaw open and facilitating intubation with the laryngoscope, and in other subsequent operations. The blocks are an important element of the present invention in that in addition to keeping the jaw open and releasing the technicians' hands for other purposes, they also help keep the guard in place as the jaw tends to clamp down on them.

The dental guard covers the incisors and other maxillary teeth, and much more effectively distributes and dissipates the shock of contact that arises during manipulation of the laryngoscope across several teeth. Such mouthguards have proven their value many times over in violent sports such as football or boxing. By distributing and dissipating the shock over several teeth, the dental guard of the present invention is more effective in preventing damage than the modifications of laryngoscopes described the prior art, which still confine the contact and stress to as few as one or two teeth.

The dental guard is preferably constructed of a suitable plastic. If desired, a plastic that may be autoclaved may be used, so that the guard could be re-used subsequently for other patients, or alternatively, the entire guard when formed as a single piece of injection-molded plastic may be so inexpensive as to make disposal after a single use economically feasible. Disposable dental guard devices may be the most preferred embodiment of the present invention, in view of the labor and energy costs associated with sterilization procedures.

The dental guard of the present invention is not intended to be fixed to the patient's teeth using any sort of adhesive, but rather will fit snugly based on the suitably resilient nature of the guard to the teeth as they grip the teeth on both sides, thereby avoiding the chance of introduction of said adhesive substances into the patient that is encountered in some of the prior art.

In each of the embodiments of the present invention, the dental guard may be quickly and easily secured in the patient's mouth, requiring minimal additional operator time as compared with a conventional laryngoscope blade. Additionally, upon completion of the intubation procedure, the dental guard of the present invention may be removed, or left in place as appropriate, where it can serve as a bite block to protect any of several tubes for directed either to the trachea or the esophagus, including an esophogeal obturator.

The dental guard is preferably constructed of a resilient plastic or other elastomeric material, capable of limited compression, and yet capable of maintaining its basic structural form. The material of construction should be a grade of plastic, rubber, or the like, innocuous to human health, and consistent with Food and Drug Administration and other regulatory agency requirements. Preferably, the dental guard would be supplied as a disposable, single-use item. The dental guard is preferably shaped to provide surface continuity with maxillary teeth when it is secured in position for use. Furthermore, the dental guard can be manufactured in different sizes, configured to different size jaws, such as a smaller size suitable for infants and young children, as well as larger sizes for adolescents and adults.

Finally, the dental guard may incorporate a conventional light, or an LED, or a fiber-optic light mounted interior to or adjacent to one of the trapezoidal blocks, and directing its light to the back of the mouth to facilitate the physician's or paramedic's examination and manipulations.

### OBJECTS OF THE INVENTION

There is a need to reduce the likelihood of trauma to which all of the mouth and throat are exposed during the technique of tracheal intubation. Therefore, one object of the present invention is to provide a device to allow intubation while adequately protecting the teeth from the handle or blade of a laryngoscope during orotracheal intubation procedures.

Because paramedics and other medical personnel often have personal preferences as to the style of laryngoscope that they routinely employ for intubation, another object of the present invention is to provide a device that may be employed to protect the teeth for all types of laryngoscopes, whether the blades are curved, partially curved, or straight.

Still another object of the present invention is to provide a device that will permit rapid and easy protection of the teeth, tongue, and oral mucosa from the blade of a laryngoscope during intubation, while requiring minimal additional actions on the part of the user.

Still another object of the present invention is to provide a device that will afford an improved degree of protection of teeth by distribution and dissipating contact stress over several teeth during an aggressive intubation procedure.

These and other objects will be better understood when the device and method of the present invention are described in the detail of the preferred embodiment and particularly pointed out and distinctly claimed in the claims.

### BRIEF DESCRIPTION OF THE DRAWINGS

FIG. 1 shows the frontal view of the dental guard according to the present invention.

FIG. 2 shows the posterior quarter view of the dental guard and features the location of the trapezoidal blocks for keeping the jaw open and for keeping the guard in place.

FIG. 3 shows a detail of the left trapezoidal block and the incorporation of a channel to hold a conventional light or LED and a miniature battery for illumination.

FIG. 4 shows the location of the dental guard as employed while the patient is undergoing an orotracheal intubation with a laryngoscope.

### DETAILED DESCRIPTION OF THE INVENTION

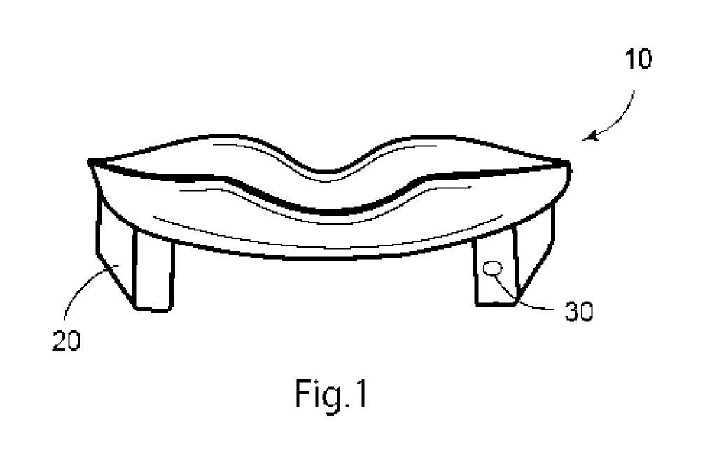

An overview of a preferred embodiment of the present invention is illustrated in FIG. 1. The inventive dental guard comprises a mouthguard 10 that covers and grips the incisors and other maxillary teeth as far back as the molars, and also comprises trapezoidal blocks or wedges 20 attached or fused to the mouthguard. The location of an optional light emitting diode LED or other light source 30 is included in the left wedge.

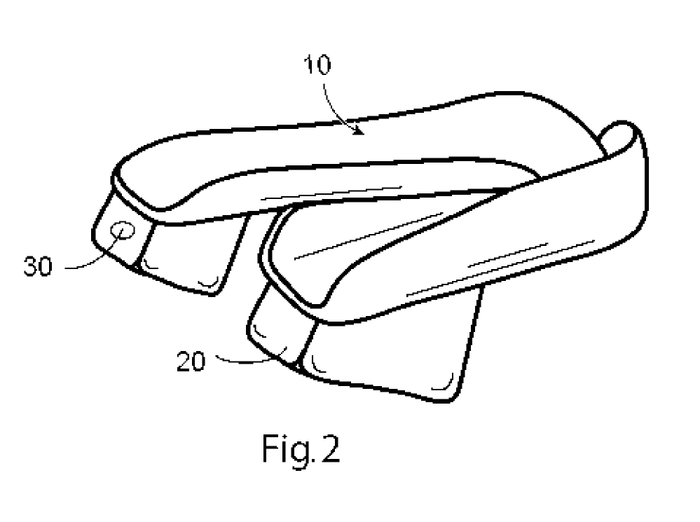

FIG. 2 provides an alternative view of the dental guard from the right posterior perspective, illustrating the trough nature of the mouthguard 10, and further illustrating the location of the trapezoidal blocks 20 with respect to the guard. The trapezoidal block on the left side in a preferred embodiment may include a channel or interior cavity to contain a miniature light 30 such as a conventional light or an LED, and its power source 40, typically a battery. This light is directed to the back of the mouth, to provide additional lighting for the benefit of the paramedic or other medical personnel.

The left and right side trapezoidal blocks 20 may be manufactured separately from the mouthguard 10 and assembled with the mouthguard by suitable means in a later manufacturing step. However, the blocks and mouthguard are formed preferably as part of the dental guard as a whole, as in a single injection-molded casting. Thus, the trapezoidal blocks are fused to the mouthguard as a continuous single piece with the advantages of simplifying manufacturing and resulting in a more durable device.

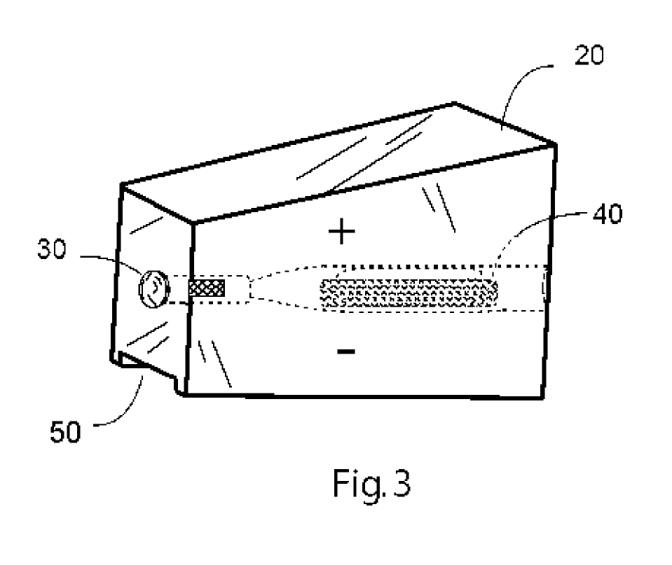

FIG. 3 illustrates a detailed view of the left side trapezoidal block 20, including a channel or cavity, formed to contain a light 30 and miniature battery 40, also shown. This light is most advantageous when used in one block, preferably the left block as shown in FIG. 2, because standardized training and procedures for medical personnel instructs them to use their left hand for the manipulation of the laryngoscope, which is introduced from the right side of the patient's mouth. Therefore the right side trapezoidal block is formed in the geometry symmetric to the left side, but may omit the channel or cavity as well as the light and battery of the left side block. The view of the lighting device 30 and battery 40 in FIG. 3 is not intended to limit the invention to the specific design of lighting device shown, but rather is intended to include any lighting device commonly known in the prior art, of suitable size and properties.

It is intended that the description of the trapezoidal blocks of the present invention not be limited to strictly trapezoidal three-dimensional geometry, but that by trapezoidal the invention is intended to embrace variations for ergonomic purposes such as rounded or smoothed edges and corners. In the same spirit it is intended that other variations from strictly trapezoidal geometry, which would otherwise formally require parallel opposite sides, be included and embraced by the present invention, so that the form of the wedge would include geometries of quadrilateral solids or other solid polygonal, elliptical, or round configurations.

Additionally, FIG. 3 shows an optional detail of the lower surface, corresponding to the surface that the mandibular molars rest against, of the trapezoidal blocks 20 that includes a shallow channel 50 to align the lower jaw and help prevent lateral motion of the lower jaw by containing the mandibular molar teeth within said channels. Preferably both trapezoidal blocks are formed with this shallow channel.

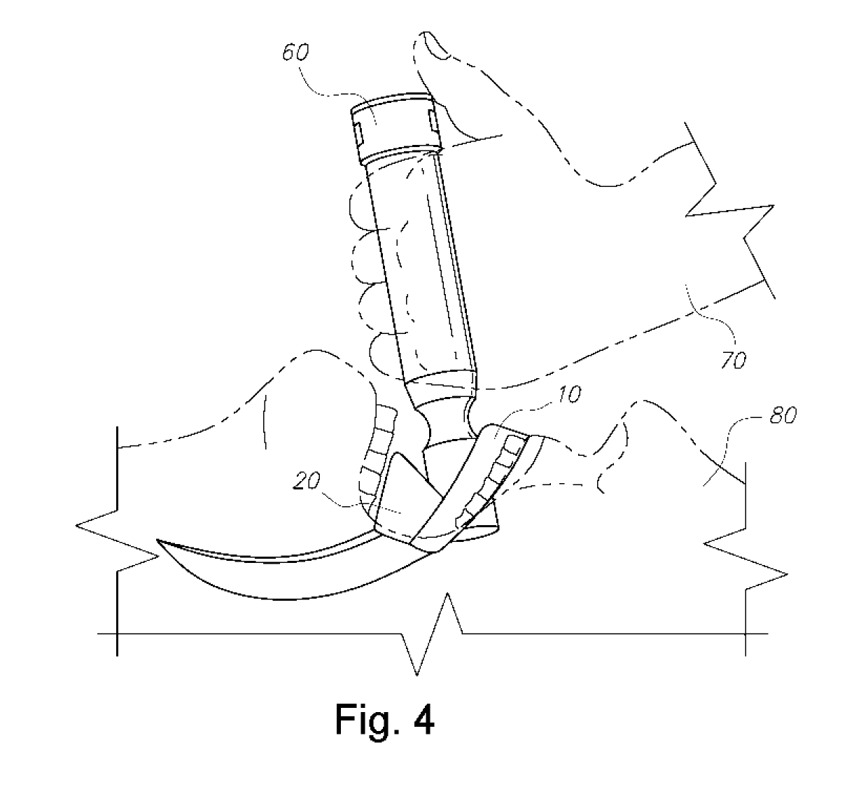

FIG. 4 illustrates the use of the dental guard, in which the guard had been installed by the paramedic 70 or other medical personnel immediately prior to the introduction of the laryngoscope 60. In the process of aligning the laryngoscope and lifting the jaw and tongue of the patient out of the way, the medical personnel 70 often leverages the laryngoscope against the maxillary incisor teeth, which can result in the trauma to said teeth that this invention seeks to prevent. Thus, by fitting the dental guard 10 over the maxillary teeth, its structure and resilient nature can distribute and dissipate the forces of the laryngoscope manipulations over several teeth and the maxillary bones.

The present invention may be used in conjunction with any of the many designs of laryngoscopes, whether they use curved blades, straight blades, or straight blades with curved tips. It is also the intention of the present invention that it may be used with various designs of laryngoscopes that include as part of said laryngoscopes resilient structures intended to offer protection against trauma to teeth.

According to the present invention, the dental guard 10 is preferably constructed of a resilient, pliable material that would maintain its structural integrity, but would absorb and diffuse mechanical stress that might be imparted to the teeth or other body structures at risk from the rigid laryngoscope. Resilience in the context of the present invention is a quality of the construction or composition of the dental guard that allows it to provide a cushioning effect to adjacent structures. More preferably, the dental guard according to the present invention provides a cushioning structure which is compressible when mechanical force is applied, yet capable of elastic reformation upon relaxation of the compressing force. Most preferably, the dental guard according to the present invention has sufficient elasticity to provide a localized cushioning effect while possessing sufficiently firm durometer strength to substantially maintain its basic shape and form.

The dental guard according to the present invention, particularly where disposable use is not practiced, would optionally be formed of material capable of withstanding sterilization by irradiation, gas or chemical agents, or steam autoclave exposure without suffering structural degradation.

A dental guard according to the present invention may be formed of a solid structure. Alternately, a dental guard may be formed of a laminar, foam, matrix, or partially hollow structure. The dental guard may be constructed of rubber, plastic, or other polymers. Including materials selected from natural or synthetic rubbers, elastomers, polyisoprenes, polyurethanes, vinyl plastisols, acrylic polyesters, polyvinylpyrrolidone-polyurethane interpolymers, butadiene rubbers, styrene-butadiene rubbers, rubber lattices, and other polymers or materials with similar resilience and pliability qualities.

In a preferred embodiment of the present invention, the dental guard may be fabricated in a molding process. Alternately, the dental guard may be fabricated by machining from a block of raw material. In a more preferred embodiment according to the present invention, the dental guard may be fabricated by an extrusion molding process. Most preferably, the dental guard may be formed by injection molding techniques. In some embodiments of the present invention, the dental guard may be fabricated with a surface coating to reduce water absorption and surface friction along the surfaces.

In a preferred embodiment according to the present invention, the dental guard 10 would be supplied as a sanitized pre-packaged, disposable item intended for single use.

Although the foregoing embodiments of the present invention have been described in some detail by way of illustration and example for purposes of clarity and understanding, it will be apparent to those skilled in the art that certain changes and modifications may be practiced within the spirit and scope of the present invention. Therefore, the description and examples presented herein should not be construed to limit the scope of the present invention, the essential features of which are set forth in the appended claims.

What is claimed is:

1. A dental guard formed of a resilient material for protecting teeth from trauma arising from the manipulation of laryngoscopes during emergency and surgical endotracheal intubation procedures comprising:
a mouthguard suitable for covering and protecting maxillary teeth from impact from the rigid structures of the laryngoscope and;
two approximately trapezoidal shaped blocks fused or attached to the posterior ends of said mouthguard substantially transverse to the location of molar teeth, said blocks suitable for keeping the jaw open and for aligning and preventing lateral motion of the lower jaw.

2. The dental guard of claim 1 wherein said trapezoidal blocks include shallow channels along the lower surface suitable to fix onto the molars of the lower jaw and further restrict lateral movement of the jaw.

3. The dental guard of claim 1 wherein said trapezoidal blocks include a channel or cavity located through the block approximately parallel to the plane of the mouthguard and directed to the rear, suitable for inclusion of a miniature light or LED and its power source, such as a battery.

4. The dental guard of claim 2 wherein said trapezoidal blocks include a channel or cavity located through the block approximately parallel to the plane of the mouthguard and directed to the rear, suitable for inclusion of a miniature light or LED and its power source, such as a battery.

5. The dental guard of claim 3 wherein said trapezoidal blocks include a miniature light or LED and its power source, such as a battery contained with said channel or cavity.

6. The dental guard of claim 4 wherein said trapezoidal blocks include a miniature light or LED and its power source, such as a battery contained with said channel or cavity.

7. The dental guard of claim 1 wherein said mouthguard and said blocks are formed in a single casting process by extrusion or injection molding techniques.

8. The dental guard of claim 2 wherein said mouthguard and said blocks are formed in a single casting process by extrusion or injection molding techniques.

9. The dental guard of claim 3 wherein said mouthguard and said blocks are formed in a single casting process by extrusion or injection molding techniques.

10. The dental guard of claim 4 wherein said mouthguard and said blocks are formed in a single casting process by extrusion or injection molding techniques.

11. The dental guard of claim 5 wherein said mouthguard and said blocks are formed in a single casting process by extrusion or injection molding techniques.

12. The dental guard of claim 6 wherein said mouthguard and said blocks are formed in a single casting process by extrusion or injection molding techniques.
